(19) United States
(12) Patent Application Publication
(10) Pub. No.: US 2015/0290140 A1
(43) Pub. Date: Oct. 15, 2015

(54) COMPOSITIONS COMPRISING MICROPARTICLES AND PROBIOTICS TO DELIVER A SYNERGISTIC IMMUNE EFFECT

(71) Applicant: NESTEC S.A., Vevey (CH)

(72) Inventors: Anurag Singh, Ecublens (CH); Zeynel Deniz Gunes, Lausanne (CH); Chiara Nembrini, Lausanne (CH); Adam Burbidge, Arzier (CH); Annick Mercenier, Bussigny (CH); Martinas Kuslys, Grosshoechstetten (CH)

(21) Appl. No.: 14/439,560

(22) PCT Filed: Oct. 30, 2013
(86) PCT No.: PCT/EP2013/072662

§ 371 Date: Apr. 29, 2015

Related U.S. Application Data
(60) Provisional application No. 61/720,197, filed on Oct. 30, 2012.

(51) Int. Cl. A61K 9/50; A61K 36/062; A61K 36/06; A61K 35/745; A23L 1/30; A61K 35/747; A61K 35/744; A61K 35/742; A61K 45/06; A61K 36/064; A61K 35/741

(52) U.S. Cl. CPC A61K 9/5036; A61K 36/064; A61K 36/062; A61K 36/06; A61K 35/745; A61K 35/741; A61K 35/747; A61K 35/744; A61K 35/742; A61K 45/06; A61K 9/5089; A23L 1/3014; A23L 1/3016; A61K 2035/115

(57) ABSTRACT

Microparticulate compositions and nutritional compositions containing the microparticulate compositions are disclosed herein. In a general embodiment, the microparticulate compositions include one or more probiotics and have a size ranging from about micron to about 20 microns. The microparticulate compositions can be added to any suitable nutritional composition.

### BACKGROUND

The present disclosure generally relates to microparticulate compositions. More specifically, the present disclosure relates to microparticulate compositions including a probiotic and/or a non-replicating probiotic and nutritional compositions containing the microparticulate composition to deliver an enhanced immune-boosting effect.

While numerous studies have highlighted the health related benefits of probiotics, the choice of strain used, industrialization process and the application of live probiotics to certain food matrices and their associated shelf life can become a limiting step. Also, orally consumed probiotics, when ingested, may depend on the numbers of probiotic bacteria delivered and the efficient interaction with the host to obtain a health related benefit. This also holds true for prebiotics and non-replicating probiotics.

The mechanism of how probiotics and prebiotics exert an effect on the host immune system is currently an area of intensive scientific research. It has been established that bacterial cell surface associated molecules are recognized by the gut mucosal immune system. These cell surface components correspond to microbial associated molecular patterns (“MAMPs”) and are known to bind to specific receptors, the pattern recognition receptors (“PRRs”), which are expressed by many immune cells and tissues such as the gut epithelium. The binding of MAMPs to PRRs is one of the mechanisms by which probiotic bacteria can elicit innate and adaptive immune responses in the host. Compositions that enable targeting of probiotics, prebiotics and non-replicating probiotic formulations to their intended site of action and allow for faster and more efficient uptake by gut resident immune cells such as dendritic cells (DCs) and M-cells can be advantageous to the consumer in delivering an enhanced immune benefit.

### SUMMARY

The present disclosure provides microparticulate compositions and nutritional compositions including a microparticulate composition made up of oil-in-water chitosan based capsule dispersions.

Alternatively, the microparticulate composition can be derived from certain other particulate preparations e.g. chitin and beta glucan. In a general embodiment, the microparticulate compositions include a probiotic and have a size ranging from about 1 micron to about 20 microns. In another embodiment, the microparticulate compositions include a non-replicating probiotic or a prebiotic and have a size ranging from about 1 micron to about 20 microns. The microparticulate compositions allow for an alternative way to deliver immunologically relevant components of viable and non-viable probiotic bacteria in different food matrices in a size (e.g., 1-20 microns) that favors an efficient uptake by immune cells in the gut. The microparticulate compositions further render probiotics, prebiotics and/or non-replicating probiotics more immunogenic as well. In another embodiment, the microparticles preparation and probiotic and/or non-replicating probiotic and/or prebiotic can also be delivered individually in a composition where they synergistically combine to deliver an enhanced immune boosting effect.

In any embodiments disclosed herein, the microparticulate compositions can have a size ranging from about 1 microns to about 20 microns. Alternatively, the microparticulate compositions can have a size ranging from about 5 microns to about 16 microns. The particles do not necessarily have a grossly spherical shape, but can be elongated ones, or even of fiber type, depending on the process used for generating them.

In any embodiments disclosed herein, there can be in each composition of chitosan based microparticle and probiotics and/or non-replicating probiotic a ratio of 1:20 (microparticles:probiotics). Alternatively, this ratio can be 1:5 or 1:10 in the microparticulate and probiotic or non-replicating probiotic composition.

In any embodiments disclosed herein, there can be in each composition of chitosan based microparticle and probiotics or non-replicating probiotic, a range of concentration of 10⁵-10¹² microparticles. Alternatively, this concentration can be in the range of 10⁷-10¹⁰ microparticles.

In any embodiment disclosed herein, the microparticulate composition made up of chitosan based microparticles can also be delivered individually in a composition along with probiotics and prebiotics added separately to deliver the synergistic immune effect. In any embodiments disclosed herein, the probiotic can be yeast such as Saccharomyces, Debaromyces, Candida, and Pichia or a combination thereof. Alternatively, the probiotic can be a mould such as Aspergillus, Rhizopus, Mucor, Penicillium or a combination thereof. The probiotic can also be a bacterium such as Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Faecalibacterium Akkerhansia, Oenococcus, Lactobacillus or any combinations thereof.

In any embodiments disclosed herein, the microorganism can be Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus, Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, Staphylococcus xylosus or any combinations thereof.

In any embodiments disclosed herein, the prebiotic can be oligosaccharides, fructooligosaccharides, galactooligosaccharides, soy, pea, oat, asparagus, artichokes, onions, wheat, chicory, pectin, guar gum, gum Arabic or any combinations thereof.

In another embodiment, the present disclosure provides a nutritional composition including one or more microparticulate compositions containing a probiotic and/or a non-replicating probiotic and having a size ranging from about 1 micron to about 20 microns. It is meant here that at least 20% of the volume of particles consists of particles having a size in the mentioned range. Every time a size range will be mentioned, it will be meant that 20% of the volume of particles consists of particle sizes falling in that range. The microparticulate compositions can also include a prebiotic and/or a non replicating probiotic. The nutritional composition can be any suitable edible composition such as a pharmaceutical composition in the form of a pill, suspension, capsule or sachet, a powdered beverage, a ready-to-drink beverage, a pet food composition, a food supplement, an infant formula, a confectionery, a chocolate product, a food product or any combinations thereof.

In any embodiments disclosed herein, the nutritional composition can include one or more of a protein, a fat and/or a carbohydrate. In addition, the nutritional composition can include one or more of a vitamin and/or a mineral.

In an alternative embodiment, the present disclosure provides a method for treating immune disorders, for immune-compromised subjects and/or for individuals suffering from skin, respiratory or food allergy. The method comprises administering to the subject in need of same a microparticulate composition comprising a probiotic and having a size ranging from about 1 micron to about 20 microns. The microparticulate composition can further include a prebiotic and/or a non replicating probiotic.

In another embodiment, the present disclosure provides a method of making a microparticulate composition. The method comprises providing a suspension of particles with an upper limit of the size distribution that is greater than 20 microns and including a probiotic and/or a non-replicating probiotic, and passing the suspension through a filter with pore size of 20 microns.

In yet another embodiment, the present disclosure provides a method of making a microparticulate composition. The method includes providing a suspension of particles with an upper limit of the size distribution that is greater than 20 microns and including a probiotic and/or a non-replicating probiotic, and performing centrifugation and re-dispersion steps, in order to form microparticulate compositions having a size ranging from about 1 micron to about 20 microns. The microparticulate compositions can then be added to a suitable nutritional composition.

An advantage of the present disclosure is to provide an improved microparticulate composition having a probiotic and/or a prebiotic and/or a non-replicating probiotic.

Another advantage of the present disclosure is to provide an improved nutritional composition including a microparticulate composition made up of chitosan based capsules containing a probiotic and/or a non-replicating probiotic and/or a prebiotic and having a size ranging from about 1 micron to about 20 microns.

Yet another advantage of the present disclosure is to provide an improved method of making a microparticulate composition including a probiotic and/or a prebiotic and having a size ranging from about 1 micron to about 20 microns.

Additional features and advantages are described herein, and will be apparent from, the following Detailed Description.

### BRIEF DESCRIPTION OF THE FIGURES

FIG. 1 shows a microscopic image of chitosan-based microcapsules in accordance with an embodiment of the present disclosure.

FIG. 2 shows a schematic of a capsule formation process including steps (a)-(c) in accordance with an embodiment of the present disclosure.

FIG. 3 illustrates the surprising synergistic immune effect of the combination of microparticles and probiotics in a immune function peripheral blood mononuclear cell assay in accordance with an embodiment of the present disclosure, where “MP” represents microparticles.

FIG. 4 illustrates the enhanced immune boosting profile in a healthy host that is delivered by a combination of chitosan microparticles and probiotics in accordance with an embodiment of the present disclosure, where “MP” represents microparticles.

FIGS. 5A-C illustrates the mechanism of action including steps (A)-(C) of chitosan-based microparticle preparations in combination with a probiotic, a prebiotic and/or a non-replicating probiotic.

### DETAILED DESCRIPTION

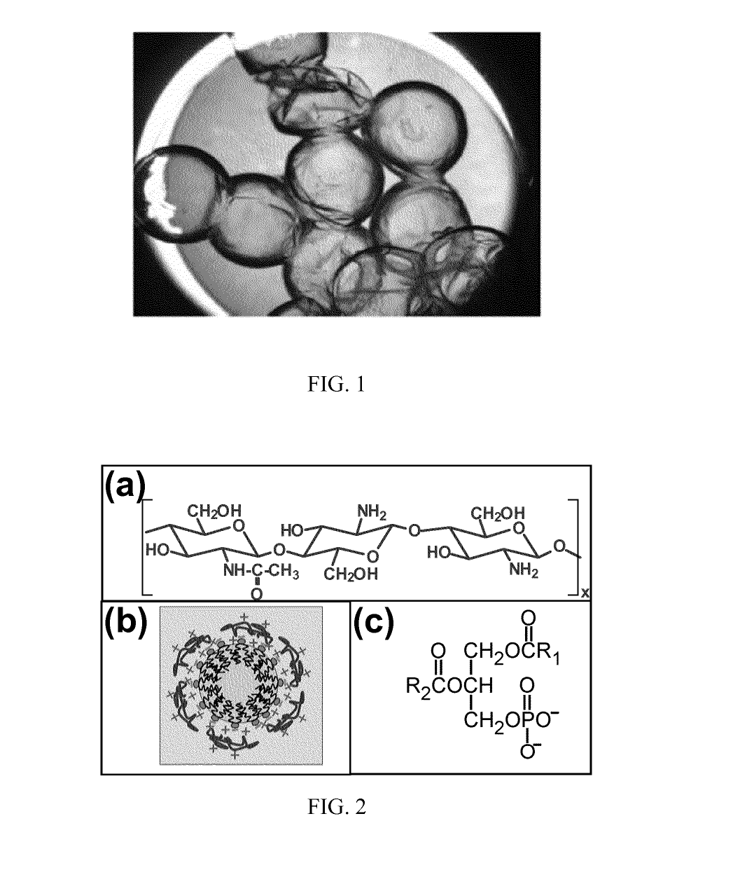

The present disclosure relates to microparticulate compositions and nutritional compositions containing the microparticulate compositions. In a general embodiment, and as shown, for example, in FIG. 1, the microparticulate compositions include one or more probiotics and have a size ranging from about 1 micron to about 20 microns. The microparticulate compositions enhance the biological effects of the probiotic and prebiotic ingredients via microparticulate formulations that can comprise a size range setting by use of, classic emulsification methods such as microfluidization or rotor-stator shearing, or by use of milling techniques in the case of stiff particles. In another embodiment, the combination of chitosan based microparticulate formulations and probiotics can deliver synergistically an enhanced immune boosting effect on the host immune system. The material inside the particle can be chosen to be very viscous, or even a gel-type of material, if such mechanical property was desired, e.g. to avoid early breakage of the particles in their journey before their reaching their target.

Providing chitosan microparticulate (e.g., 1-20 microns) compositions of probiotic and prebiotic ingredients and the associated cell wall components will allow for their faster and more efficient uptake by gut resident cells (e.g., M cells and intestinal dendritic cells) thereby initiating a signaling cascade that can modulate the host immune system. This will result in enhanced innate and adaptive immune effects of the probiotic and prebiotic ingredients at mucosal sites over conventional probiotic nutritional formulations. Advantageously, the microparticulate compositions can be used in nutritional compositions or food supplements for immune disorders (e.g., ulcerative colitis Crohn's disease, multiple sclerosis, and arthritis), for immune-compromised subjects (e.g., immune deficiency syndrome), and for individuals suffering from allergic disorders (e.g., atopic dermatitis, food allergy symptoms, eosinophilic esophagitis, allergic rhinitis, allergic asthma).

Examples of microparticles that may be used in the present disclosure may be found in WO 2011/101415, the entire content of which is incorporated herein by reference. The microparticles of WO 2011/101415 include an oily fraction, a hydrophilic fraction, and at least one body having a shell comprising several layers of chitosan and at least one lipidic phosphatidic acid surfactant, and a content comprising an internal phase containing a hydrophilic component and/or a hydrophobic component. The lipidic phosphatidic acid surfactant may be, for example, an ammonium phosphatidic fatty acid, or a mixture of phosphatidic acids comprised in lecithin (e.g., lecithin YN). Accordingly, the microparticles of the present disclosure may be liquid-filled chitosan-lipidic phosphatidic acid surfactant particles with probiotics.

In any embodiments disclosed herein, the microparticulate composition has a size (e.g., overall thickness, length, width, diameter) ranging from about 1 micron to about 20 microns. More specifically, the size can be about 1, 2, 3, 4, 5, 6, 7, 8, 9, 10, 11, 12, 13, 14, 15, 16, 17, 18, 19, 20 microns and the like. It should be appreciated that any two sizes of the microparticulate composition recited herein can further represent end points in a preferred range of sizes. For example, the size of about 5 microns and about 15 microns can represent the individual sizes of the microparticulate composition as well as a preferred range of the size of the microparticulate composition ranging from about 5 microns to about 15 microns.

As used herein, the term “probiotic” means microbial cell preparations or components of microbial cells with a beneficial effect on the health or well-being of the host. See Salminen S, Ouwehand A. Benno Y. et al “Probiotics: how should they be defined” Trends Food Sci. Technol. 1999:10 107-10, which is incorporated herein by reference. The probiotics can be replicating or non-replicating probiotics. “Non-replicating” probiotics are further defined as probiotic bacteria which have been heat treated. This includes microorganisms that are inactivated, dead, non-viable and/or present as fragments such as DNA, metabolites, cytoplasmic compounds, and/or cell wall materials.

Examples of suitable probiotic micro-organisms include yeasts such as Saccharomyces, Debaromyces, Candida, and Pichia, moulds such as Aspergillus, Rhizopus, Mucor, Penicillium and bacteria such as the genera Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Faecalibacterium Akkerhansia, Oenococcus and Lactobacillus.

Specific examples of suitable probiotic micro-organisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus, Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

In any embodiments disclosed herein, there can be a ratio of microparticles preparations to probiotics of about 1:10 (microparticles:probiotics) in each microparticulate composition. For example, the ratio of the composition can include about 1:1, 1:2, 1:3, 1:4, 1:5, 1:6, 1:7, 1:8, 1:9, 1:10 and the like a maximum of 1:20. It should be appreciated that any two amounts of the cells in the microparticulate composition recited herein can further represent end points in a preferred range of cell amounts.

In any embodiments disclosed herein, there can be in each composition of chitosan based microparticle and probiotics, a range of concentration of 10⁵-10¹² microparticles. Alternatively, this concentration can be in the range of 10⁷-10¹⁰ microparticles.

As used herein, the term “prebiotic” means a non-digestible food ingredient that beneficially affects the host by selectively stimulating the growth and/or activity of one or a limited number of bacteria in the colon and thus improves host health. See, Gibson and Roberfroid “Dietary Modulation of the Human Colonic Microbiota: Introducing the Concept of Prebiotics” J. Nutr 125:1401 -1412, which is incorporated herein by reference.

The prebiotics may be provided in any suitable form. Suitable prebiotics include oligosaccharides such as fructooligosaccharides and galactooligosaccharides. The prebiotic may also be provided in the form of a plant material that contains the fiber. Suitable plant materials include soy, pea, oat, asparagus, artichokes, onions, wheat or chicory, or residues of these plant materials. Other prebiotics may include pectin, guar gum, gum Arabic, and the like.

Alternatively, the prebiotic fiber may be provided as an inulin extract. Extracts from chicory are particularly suitable. Suitable inulin extracts may be obtained from Orafti SA of Tirlemont 3300, Belgium under the trade mark “Raftiline.” For example, the inulin may be provided in the form of Raftiline® ST, which is a fine white powder that contains about 90% to about 94% by weight of inulin, up to about 4% by weight of glucose and fructose, and about 4% to 9% by weight of sucrose. Alternatively, the fiber may be in the form of a fructooligosaccharide such as that obtained from Orafti SA of Tirlemont 3300, Belgium under the trade mark “Raftilose.” For example, the inulin may be provided in the form of Raftilose® P95. Otherwise, the fructooligosaccharides may be obtained by hydrolyzing inulin, by enzymatic methods, or by using micro-organisms.

The microparticulate compositions may further contain hydrocolloids (e.g., gums, proteins, modified starches), binders, film forming agents, encapsulating agents/materials, wall/shell materials, matrix compounds, coatings, surface active agents, solubilizing agents (e.g., oils, fats, waxes, lecithins etc.), adsorbents, carriers, fillers, co-compounds, dispersing agents, processing aids (e.g., solvents), flowing agents, taste masking agents, weighting agents, jellifying agents, gel forming agents, antioxidants and antimicrobials.

The microparticulate compositions may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. In all cases, such further components will be selected having regard to their suitability for the intended recipient.

The microparticulate compositions can also be added to any suitable nutritional compositions. The nutritional compositions can be any suitable composition such as a pharmaceutical composition in the form of pill, capsule, sachet, a powdered beverage, a ready-to-drink beverage, a pet food composition, a food supplement, an infant formula, a confectionery, a chocolate product, a food product or a combination thereof. The nutritional compositions may include one or more of proteins, fats, carbohydrates and any other suitable ingredient.

Fat sources include canola oil, corn oil, palm olein, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils.

Any suitable carbohydrate may be used such as, for example, sucrose, lactose, glucose, fructose, corn syrup solids, saccharose, maltodextrins, starch and mixtures thereof. Dietary fiber may also be added if desired. Dietary fiber passes through the small intestine undigested by enzymes and functions as a natural bulking agent and laxative. Dietary fiber may be soluble or insoluble and in general a blend of the two types is preferred. Suitable sources of dietary fiber include, but are not limited to, soy, pea, oat, pectin, guar gum, gum Arabic, fructooligosaccharides and galacto-oligosaccharides.

The nutritional compositions containing the microparticulate compositions can further include minerals and micronutrients such as trace elements and vitamins. Examples of minerals, vitamins and other micronutrients optionally present in the nutritional compositions include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. Minerals are usually added in salt form.

The nutritional compositions containing the microparticulate compositions can include one or more food grade emulsifiers such as, for example, diacetyl tartaric acid esters of mono- and di-glycerides, lecithin and mono- and di-glycerides. Similarly suitable salts and stabilisers may be included.

In an alternative embodiment, the present disclosure provides a method of treating an immune related illness in a subject. This illness can be related to any condition where delivering an enhanced immune benefit effect with microparticulate formulations comprising probiotic would either lower the risk of developing a disease or alleviate the symptoms of the illness. Such immune related conditions can range from (but not limited to) allergic disorders (e.g., atopic dermatitis, food allergy symptoms, eosinophilic esophagitis, allergic rhinitis, allergic asthma) to inflammatory disorders (e.g., ulcerative colitis Crohn's disease, multiple sclerosis, arthritis, immune deficiency syndrome). The method comprises administering to the subject in need of same a microparticulate composition made up of chitosan and comprising a probiotic or non-replicating probiotic and having a size ranging from about 1 micron to about 20 microns. The microparticulate composition can further include a prebiotic. The microparticulate composition can be added to a suitable nutritional composition and administered to the subject in any suitable manner

In an alternative embodiment, the present disclosure provides a method of delivering an enhanced immune boosting effect in an immune-depressed individual, an elderly individual, a critically ill individual, a hospitalized subject, or a surgery patient.

In any embodiments disclosed herein, there can be in each nutritional composition of chitosan based microparticle and probiotics and/or non-replicating probiotic, a range of concentration of 10⁵-10¹² microparticles. Alternatively, this concentration can be in the range of 10⁷-10¹⁰ microparticles.

In any embodiments disclosed herein, there can be in each nutritional composition of chitosan based microparticle and probiotics and/or non-replicating probiotic, a final concentration of 0.05%-5% by weight of the nutritional composition.

In an alternative embodiment, the present disclosure provides a method of delivering an enhanced immune boost to a healthy subject, resulting in prevention from any unwanted acute or chronic immune related disorders.

The microparticulate compositions can be made using suitable techniques described in the literature, which will depend on the type of particle and size range to be produced. A schematic example of the capsule formation process is provided in FIG. 2, which includes (a) a generic chemical structure of chitosan with deacetylation degree DA; (b) a sketch of the first layer of chitosan/PFacidYN complexation at the interface of an oil-in-water drop; and (c) a generic chemical structure of phosphatidic fatty acid molecules.

For the chitosan-based microparticles, an exemplary method of formation is described in Example 1 of this document, and comprises an emulsification step. For chitin or beta-glucan based particles, milling or micro-milling processes may be used. In an embodiment, the method includes providing a suspension of particles having a size greater than 20 microns and including a probiotic, a subsequent step allows narrowing down the size distribution to a range 1-20 micron. The step of narrowing down the size distribution may be performed either by filtration through filters of appropriate pore size (e.g., 20 micron pore size filter to remove the larger particles; 1 micron pore size filter to keep only the particles larger than 1 micron), and/or by using centrifugation-re-dispersion steps. When a centrifugation step is performed, particles go down the centrifugation tubes according to their size and average density. In the case of chitosan based microparticles, centrifugation enables one to concentrate particles larger than 1 micron at the bottom of the tube, allowing easy removal of smaller particles. The microparticulate compositions can then be added to any suitable nutritional composition in any suitable amount.

In another embodiment, the present disclosure provides a method of making a microparticulate composition. The method comprises providing a suspension of particles having a size greater than 20 microns and including a probiotic and a prebiotic, and using the same step of narrowing down the particulate size distribution to the range 1-20 microns, as described in the preceding paragraph. The microparticulate compositions of the present disclosure can then be added to any suitable nutritional composition in any suitable amount. For example, the microparticulate compositions can be used in pharmaceutical applications, medical foods, food supplements, complete nutritional formulas, etc.

Details of a particular process for producing microparticulate compositions are disclosed in WO 2011/101415 and in “Gunes et al., Soft Matter, 7, 9206 (2011),” both of which are incorporated herein by reference.

### EXAMPLES

### Example 1

### Method for Making Microparticles

Chitosan is a carbohydrate polymer obtained from the deacetylation of chitin (poly-b-1,4-D-N-acetylglucosamine) by alkali treatment, its generic structure can be found in the literature. See, e.g., Gunes et al., Soft Matter, 7, 9206 (2011). Its structure depends on its degree of deacetylation, generally comprised between 60% and 99% (e.g., 100% deacetylation would yield poly D-glucosamine, which sets the electrostatic charge density). The ammonium phosphatidic fatty acid used in the chitosan based composition is a commercial lecithin known as lecithin YN, purchased from Palsgaard (e.g., Palgaard®4448, food-grade E442, commonly used as viscosity modifier in chocolate formulations). Lecithin YN is insoluble in water at any temperature. It is soluble in common food oils and melted fats up to several grams per litre. The main pKa values of the phosphatidic acid molecules in lecithin YN are 3.0 and 8.0, so when adsorbed at oil/water interface with pH around 3 or higher, the molecules carry a significant fraction of negative charges; that fraction is 0.25 at pH 3. At pH below the pKa of the chitosan chains, the major part of the acido-basic groups of chitosan are charged.

### Capsules Formation

1. A chitosan solution of concentration in the range 0.1-1.0% w/w was prepared by dispersion of a chitosan powder in water (average molecular weight MW typically in the range 100,000 to 500,000 g/mol, but it could be lower or higher; here it was 300,000 g/mol), deacetylation degree in the range 80%. Chloride acid was used to achieve proper dissolution, to set the pH at 3. In the present example, the chitosan concentration was 0.24% w/w in water. The pH was 3 for the capsule fabrication.

2. Lecithin YN from was dissolved in mid-chain-triglyceride oil at a concentration in the range 0.1-0.5% w/w. The lecithin YN concentration was 0.5% w/w in oil.

3. An emulsion was formed by dispersing the lecithin YN oil solution formed in step 2 in water at pH 3, for oil to water volume proportion typically in the range 1.0% - 40%, using a mechanical dispersion method, typically a high-speed rotor-stator. In this example, the method used was this one described just above.

4. The emulsion formed in step 3 was mixed with the chitosan solution prepared in step 1 in 1:1 weight proportions, by soft mechanical mixing.

5. The dispersion formed in step 4 was left in quiescent state or kept under gentle mechanical stirring, which leaves the interfacial shell to grow in thickness, for 4 days in the present example.

6. Several size refinement steps were employed:

6.1 Filtration through a filter of 20 μm pore size was done. The permeate was kept and processed further.

6.2. Two centrifugation-re-dilution steps at 2000 m·s⁻² g were done. After each centrifugation step, the supernatant, which was poor in capsules, was removed. The re-dilution steps were done using Millipore water. The final pH value was adjusted to 7 using sodium hydroxide (0.01 mol/L). The final particle concentration and the capsule sizes were comprised in the range 1-20 μm, with a maximum probability at 10 μm. The particulate dispersion obtained may be spray-dried further on.

### Examples 2 & 3

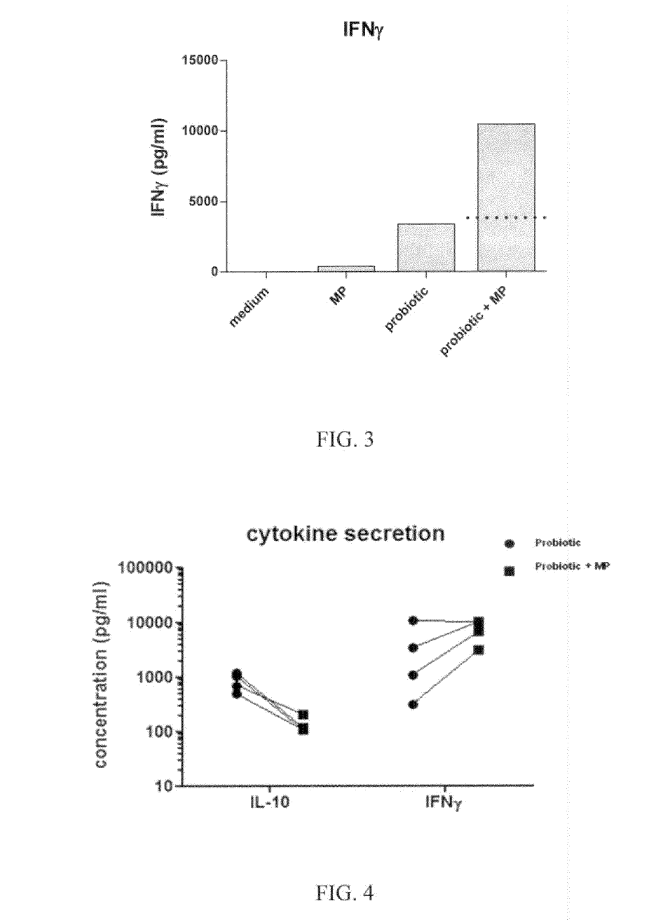

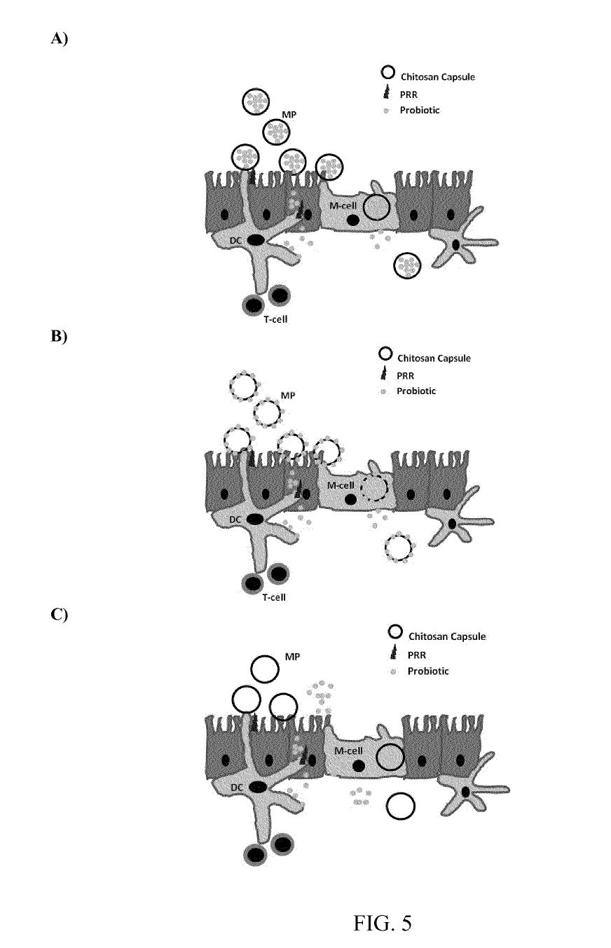

Applicants have surprisingly and unexpectedly found that a combination of chitosan microparticles and a probiotic strain is synergistic and enhances IFN-γproduction (Example 2) from peripheral blood mononuclear cells (“PBMCs”). PBMCs from four different healthy volunteers were obtained from blood filters that were received from the local transfusion facility. The blood was diluted in HBSS (“Hank's Balanced Salt Solution”). The diluted blood was then layered over a Histopaque-1077 gradient and centrifuged at 500 g for 20 min at room temperature. PBMC were harvested from the interface layer, washed twice with HBSS and then counted. PBMC concentration was adjusted to 1.5×10⁶ viable cells/ml. Five hundred microliters of a 1×10⁶ cell suspension were cultured with the treatments at 37° C. with 5.0% CO2 in 48-well plates. All treatments were performed in quadruplicates. Following a 36 hour incubation, the supernatant fractions were harvested for ELISA analysis. Cytokines were measured in cell culture supernatants by a multiplex kit. As shown by FIG. 3, compared to microparticles (“MP”) or probiotic stimulation alone, the combination of chitosan microparticulate formulations and the probiotic strain induced a two-fold higher immune response (increase in IFN-γ production). The resulting immune profile with the combination of chitosan microparticles and probiotics compared to MP or probiotic stimulation alone is also an ideal immune-boosting profile (Example 3) as it also lowers the production of an immune-suppressive cytokine IL-10 and at the same time results in an enhanced production of IFN-γ. This effect is clearly shown in FIG. 4. Such a profile may be beneficial in immune-compromised subjects (e.g., immune deficiency syndrome, cancer).

### Example 4

Compositions containing chitosan based microparticle capsule dispersions with probiotics (or alternatively prebiotics, non-replicating probiotics) can be orally administered to a subject to deliver an immune boosting effect. Such a composition allows for an efficient uptake of probiotics and microparticles at the interface of the gut mucosal immune system by specialized cells such as M-cells and dendritic cells (“DCs”) that eventually activates pattern recognition receptors (“PRRs”) on these cells and triggers a synergistic immune response that is more robust compared to probiotics or microparticles administered alone. As shown in FIG. 5, the resulting compositions containing chitosan based microparticle capsule dispersions with probiotics (or alternatively prebiotics and/or non-replicating probiotics) can be delivered in three different ways resulting in the synergistic immune effect:

A) Chitosan capsules (size of, for example, 5-20 microns) containing probiotics and/or non-replicating probiotics and/or prebiotics in the inside that allows them to reach the site of action (enable targeting and allows faster and more efficient uptake by gut resident immune cells such as DCs and M-cells). See, e.g., FIG. 5A.

B) Chitosan capsules (size of, for example, 5-20 microns) containing probiotics and/or non-replicating probiotics and/or prebiotics on the outside layers due to the high affinity of chitosan. See, e.g., FIG. 5B.

C) Compositions containing chitosan microparticles or capsules and probiotic and/or non-replicating probiotics separately in the matrix. The two delivered separately in a combined composition act synergistically to deliver an enhanced immune effect to the host. See, e.g., FIG. 5C.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A microparticulate composition comprising at least one microparticle and a probiotic and having a size ranging from about 1 micron to about 20 microns.

2. The microparticulate composition of claim 1, wherein the microparticulate composition has a size ranging from about 5 microns to about 15 microns.

3. The microparticulate composition of claim 1, wherein the microparticulate composition has a size ranging from about 8 microns to about 12 microns.

4. The microparticulate composition of claim 1, wherein the microparticulate composition comprises one of chitosan based capsules and particles based on macromolecules having a molecular composition similar to chitosan polysaccharide.

5. The microparticulate composition of claim 1 comprising a ratio of microparticle to probiotic of from about 1:1 to about 1:20.

6. The microparticulate composition of claim 1 comprising a ratio of microparticle to probiotic of from about 1:2 to about 1:10.

7. The microparticulate composition of claim 1, wherein the probiotic is a yeast selected from the group consisting of Saccharomyces, Debaromyces, Candida, Pichia and combinations thereof.

8. The microparticulate composition of claim 1, wherein the probiotic is a mold selected from the group consisting of Aspergillus, Rhizopus, Mucor, Penicillium, and combinations thereof.

9. The microparticulate composition of claim 1, wherein the probiotic is a bacteria selected from the group consisting of Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus, Lactobacillus and combinations thereof.

10. The microparticulate composition of claim 1, wherein the probiotic is a bacteria selected from the group consisting of Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, Staphylococcus xylosus and combinations thereof.

11. The microparticulate composition of claim 1 further comprising a prebiotic.

12. The microparticulate composition of claim 11, wherein the prebiotic is selected from the group consisting of oligosaccharides, fructooligosaccharides, galactooligosaccharides, soy, pea, oat, asparagus, artichokes, onions, wheat, chicory, pectin, guar gum, gum Arabic and combinations thereof.

13. The microparticulate composition of claim 1 further comprising at least one non-replicating probiotic in each microparticle.

14. A method of treating a respiratory illness in a subject in need of same, the method comprising:
administering to the subject in need of same a microparticulate composition comprising at least one microparticle and a probiotic, the microparticulate composition having a size ranging from about 1 micron to about 20 microns.

15. The method of claim 14, wherein the microparticulate composition includes a prebiotic.

16. A method of treating an immune related disorder in a subject having same, the method comprising:
administering to the subject a microparticulate composition comprising at least one microparticle and a probiotic, the microparticulate composition having a size ranging from about 1 micron to about 20 microns.

17. The method of claim 16, wherein the microparticulate composition includes a prebiotic.

18. A method of delivering an immune boost to a healthy subject to prevent diseases, the method comprising:
administering to the subject in need of same a microparticulate composition comprising at least one microparticle and a probiotic, the microparticulate composition having a size ranging from about 1 micron to about 20 microns.

19. The method of claim 18, wherein the microparticulate composition includes a prebiotic.

20. A method of alleviating the symptoms of an immune-related disorder in a subject having same, the method comprising:
administering to the subject a microparticulate composition comprising at least one microparticle and a probiotic, the microparticulate composition having a size ranging from about 1 micron to about 20 microns.

21. The method of claim 20, wherein the microparticulate composition includes a prebiotic.

22. The method of claim 20, wherein the symptoms are selected from the group consisting of allergic disorders, inflammatory disorders, and combinations thereof.

23. A method of making a microparticulate composition, the method comprising:
providing a suspension of particles having a size greater than 20 microns and comprising a probiotic; and
passing the suspension through a filter with a pore size of about 20 microns to form a microparticulate composition comprising at least one microparticle and a probiotic, the microparticulate composition having a size ranging from about 1 micron to about 20 microns.

24. The method of claim 23 further comprising adding the microparticulate composition to a nutritional composition.

25. A method of making a microparticulate composition, the method comprising:
providing a suspension of particles having a size greater than 20 microns and comprising a probiotic; and
performing centrifugation and re-dispersion to form a microparticulate composition comprising at least one microparticle and a probiotic, the microparticulate composition having a size ranging from about 1 micron to about 20 microns.

26. The method of claim 25 further comprising adding the microparticulate composition to a nutritional composition.

27. A composition comprising:
an oily fraction;
a hydrophilic fraction; and
at least one body, wherein the body comprises
a shell comprising a plurality of molecular layers of complexant molecules, the complexant molecules being chitosan and at least one lipidic phosphatidic acid surfactant, the lipidic phosphatidic acid surfactant content comprising at least 20% by weight of all lipidic surfactants present in the shell, and
a content comprising an internal phase comprising a probiotic and a component selected from the group consisting of hydrophilic, hydrophobic, and combinations thereof.

28. The composition of claim 27, wherein the composition further comprises a prebiotic.

29. The composition of claim 27, wherein the lipidic phosphatidic acid surfactant is one of an ammonium phosphatidic fatty acid and a mixture of phosphatidic acids comprised in lecithin.
